(19) United States
(12) Patent Application Publication
(10) Pub. No.: US 2004/0141930 A1
(43) Pub. Date: Jul. 22, 2004

(54) Oil-in-water oxidizing cream emulsion for treating human keratin fibers

(76) Inventor: Frederic Legrand, Courbevoie (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/690,563

(22) Filed: Oct. 23, 2003

Related U.S. Application Data
(60) Provisional application No. 60/421,074, filed on Oct. 25, 2002.

(30) Foreign Application Priority Data
Oct. 23, 2002 (FR) 02 13240

(51) Int. Cl. A61K 7/135; A61K 7/075; A61K 7/08

(52) U.S. Cl. 424/62; 424/70.24

(57) ABSTRACT

Disclosed herein is an oxidizing cream emulsion for treating human keratin fibers such as hair, comprising at least one oxidizing agent, at least one fatty alcohol chosen from (C₈-C₃₀) fatty alcohols, at least one surfactant chosen from nonionic and anionic surfactants and at least one amphiphilic polymer comprising at least one 2-acrylamido-2-methylpropanesulphonic acid unit in free form or partially or totally neutralized form and at least one hydrophobic unit comprising from 6 to 50 carbon atoms.

This application claims benefit of U.S. Provisional Application No. 60/421,074, filed Oct. 25, 2002.

Disclosed herein is an oxidizing cream emulsion for treating human keratin fibers, such as hair, comprising at least one oxidizing agent, at least one fatty alcohol chosen from (C₈-C₃₀) fatty alcohols, at least one surfactant chosen from nonionic and anionic surfactants and at least one amphiphilic polymer comprising at least one 2-acrylamido-2-methylpropanesulphonic acid unit in free form or partially or totally neutralized form and at least one hydrophobic unit comprising from 6 to 50 carbon atoms.

Further disclosed herein is the use of the at least one amphiphilic polymer to stabilize the viscosity of an oxidizing oil-in-water emulsion comprising at least one fatty alcohol chosen, for example, from (C₈-C₃₀) fatty alcohols and at least one surfactant chosen from nonionic and anionic surfactants.

Additionally disclosed are dyeing, bleaching and permanent-reshaping processes using the disclosed emulsion.

In cosmetics, in the fields of dyeing, bleaching and permanent-reshaping of human keratin fibers such as hair, it is known to use oxidizing compositions.

For instance, in the oxidation dyeing of hair, oxidizing compositions are mixed with oxidation dyes such as bases and couplers, which are colorless in themselves, to generate colored compounds and dyes by a process of oxidative condensation. Oxidizing compositions can also be used in the direct dyeing of the hair as a mixture with certain direct dyes that are colored, and coloring, in order to obtain a coloration with a lightening effect on the hair. Among the oxidizing agents conventionally used for dyeing keratin fibers, mention may be made of hydrogen peroxide and compounds capable of producing hydrogen peroxide by hydrolysis, such as urea peroxide, and persalts such as perborates and persulphates. For example, hydrogen peroxide can be used.

In hair bleaching, bleaching compositions generally comprise at least one oxidizing agent. Among the oxidizing agents, those conventionally used include, for example, hydrogen peroxide and compounds capable of producing hydrogen peroxide by hydrolysis, such as urea peroxide and persalts such as perborates, percarbonates and persulphates. For example, hydrogen peroxide and persulphates can be used.

These compositions are mainly formed from anhydrous products that comprise alkaline compounds, such as amines and alkaline silicates, and a peroxygenated reagent such as ammonium or alkali metal persulphates, perborates or percarbonates, which can be diluted at the time of use with an aqueous hydrogen peroxide composition.

Bleaching compositions can also be in the form of ready-to-use oil-in-water emulsions based on hydrogen peroxide.

As disclosed herein, the expression “ready-to-use composition” means the composition intended to be applied in unmodified form to the keratin fibers, i.e., it may be stored in unmodified form before use or may result from the extemporaneous mixing of two or more compositions.

In the permanent reshaping of the hair, in a first stage, the keratin —S—S-disulphide (cystine) bonds can be opened using a composition comprising a suitable reducing agent (reduction), followed, after having rinsed the head of hair thus treated, by reconstituting the disulphide bonds in a second stage, by applying to the hair, which has been placed under tension beforehand (curlers and the like), an oxidizing composition (oxidation, also called fixing) so as to finally give the hair the desired shape. This technique thus makes it possible either to make the hair wavy or to relax or straighten it. The new shape given to the hair by a chemical treatment such as above can be remarkably long-lasting and, for example, can withstand the action of washing with water or shampoos, as opposed to simple standard techniques of temporary reshaping, such as hairsetting.

The oxidizing compositions used in the fixing stage of the permanent reshaping as described above are usually compositions based on an aqueous hydrogen peroxide solution.

In these cosmetic applications, the oxidizing compositions can usually be formulated in the form of oil-in-water (O/W) emulsions based on fatty alcohol and nonionic or anionic surfactant, so as to optimize the application and use qualities of the dyeing, bleaching or permanent-reshaping compositions comprising them, and, for example, in order for them to have a sufficient consistency so as not to run outside the zones of the head of hair or locks of hair to be treated.

It has been found, however, that the oxidizing O/W emulsions formed from fatty alcohol(s) and from nonionic or anionic surfactant(s) can undergo substantial texture and viscosity changes over time, and that the cosmetic compositions formed therefrom can lose their usual use qualities. For example, such compositions may thicken substantially over time, thus gradually making it very difficult for hairstylists to extract them from the device containing them.

The present inventor has discovered, surprisingly, that it is possible to obtain oxidizing cream O/W emulsions whose viscosity and texture may change significantly less over time, if at least one amphiphilic polymer comprising at least one 2-acrylamido-2-methylpropanesulphonic (AMPS) acid unit in free form or partially or totally neutralized form and at least one hydrophobic unit comprising from 6 to 50 carbon atoms is introduced into the oxidizing O/W emulsion. Thus, the dyeing, bleaching or permanent-reshaping compositions comprising such emulsions can be stable and can have improved and more efficient application and use qualities.

Disclosed herein is thus an oxidizing cream oil-in-water emulsion for treating a keratin material, comprising at least one oxidizing agent, at least one fatty alcohol chosen from (C₈-C₃₀) fatty alcohols, at least one surfactant chosen from nonionic and anionic surfactants and at least one amphiphilic polymer comprising at least one 2-acrylamido-2-methylpropanesulphonic acid unit in free form or partially or totally neutralized form of formula (I) below and at least one hydrophobic unit comprising from 6 to 50 carbon atoms,

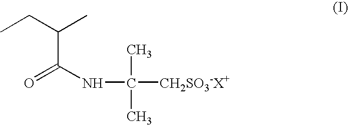

wherein X⁺is chosen from a proton, alkali metal cations, alkaline-earth metal cations and an ammonium ion.

Further disclosed herein is the use of the at least one amphiphilic polymer to stabilize the viscosity of an oxidizing O/W emulsion comprising at least one fatty alcohol and at least one surfactant chosen from nonionic and anionic surfactants.

However, other characteristics, aspects, subjects and advantages of the invention will emerge even more clearly on reading the description and the examples that follow.

As disclosed herein, the at least one amphiphilic polymer comprises at least one 2-acrylamido-2-methylpropanesulphonic (AMPS) acid unit in free form or partially or totally neutralized form and at least one hydrophobic unit comprising from 6 to 50 carbon atoms.

The term “amphiphilic polymer” means any polymer comprising both a hydrophilic portion and a hydrophobic portion such as a fatty chain.

The hydrophobic portion present in the at least one amphiphilic polymer disclosed herein can comprise, for example, from 12 to 22 carbon atoms.

The at least one amphiphilic polymer disclosed herein has a number-average molecular weight ranging, for example, from 1 000 to 20 000 000 g/mol, such as from 20 000 to 5 000 000 g/mol, and further such as from 100 000 to 1 500 000 g/mol.

The at least one amphiphilic polymer disclosed herein may be crosslinked or non-crosslinked. In one embodiment, crosslinked amphiphilic polymers are used.

When the at least one amphiphilic polymer is crosslinked, the crosslinking agents used may be chosen from the polyolefinically unsaturated compounds commonly used for crosslinking polymers obtained by free-radical polymerization.

Examples that may be mentioned include divinylbenzene, diallyl ether, dipropylene glycol diallyl ether, polyglycol diallyl ethers, triethylene glycol divinyl ether, hydroquinone diallyl ether, ethylene glycol di(meth)acrylate and tetraethylene glycol di(meth)acrylate, trimethylolpropane triacrylate, methylenebisacrylamide, methylenebis-methacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, other allyl or vinyl ethers of polyfunctional alcohols, and allylic esters of phosphoric and/or vinylphosphonic acid derivatives, and mixtures thereof.

Methylenebisacrylamide, allyl methacrylate, and trimethylolpropane triacrylate (TMPTA) can, for example, also be used. The degree of crosslinking ranges, for example, from 0.01 mol % to 10 mol %, such as from 0.2 mol % to 2 mol % relative to the total moles of the polymer.

The at least one amphiphilic polymer disclosed herein may be chosen, for example, from random amphiphilic AMPS polymers modified by reaction with an entity chosen from C₆-C₂₂ n-monoalkylamines and di-n-alkylamines, such as those described in patent application WO 00/31154. These polymers may also comprise at least one additional hydrophilic unit chosen, for example, from (meth)acrylic acids, β-substituted alkyl derivatives thereof and esters thereof obtained with monoalcohols or mono- or polyalkylene glycols, (meth)acrylamides, vinylpyrrolidone, maleic anhydride, itaconic acid, and maleic acid.

These same copolymers may also comprise at least one unit not comprising a fatty chain, chosen, for example, from (meth)acrylic acids, β-substituted alkyl derivatives thereof and esters thereof obtained with monoalcohols or mono- or polyalkylene glycols, (meth)acrylamides, vinylpyrrolidone, maleic anhydride, itaconic acid, and maleic acid.

These copolymers are described, for example, in patent application EP-A-750 899, U.S. Pat. No. 5,089,578 and in the following publications from Yotaro Morishima:

“Self-assembling amphiphilic polyelectrolytes and their nanostructures”, Chinese Journal of Polymer Science, Vol. 18, No. 40, (2000), pp. 323-336;

“Micelle formation of random copolymers of sodium 2-(acrylamido)-2-methylpropanesulphonate and a nonionic surfactant macromonomer in water as studied by fluorescence and dynamic light scattering”, Macromolecules, Vol. 33, No. 10, (2000), pp. 3694-3704;

“Solution properties of micelle networks formed by nonionic moieties covalently bound to a polyelectrolyte: salt effects on rheological behaviour”, Langmuir, Vol. 16, No. 12, (2000), pp. 5324-5332; and “Stimuli responsive amphiphilic copolymers of sodium 2-(acrylamido)-2-methylpropanesulphonate and associative macromonomers”, Polym. Preprint, Div. Polym. Chem., 40(2), (1999), pp. 220-221”.

The hydrophobic units of these copolymers are, for example, chosen from the acrylates and acrylamides of formula (II) below:

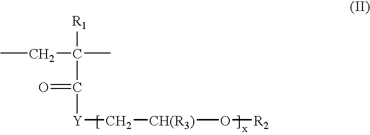

wherein: R₁ and R₃, which may be identical or different, are each chosen from a hydrogen atom and linear and branched C₁-C₆ alkyl radicals, such as a methyl radical; Y is chosen from O and NH; R₂ is a hydrophobic hydrocarbon-based radical chosen from those comprising from 6 to 50 carbon atoms such as from 12 to 22 carbon atoms; x is a number of moles of alkylene oxide and ranges from 0 to 100.

The hydrophobic hydrocarbon-based radical R₂ is, for example, chosen from linear C₆-C₁₈ alkyl radicals, for example, n-hexyl, n-octyl, n-decyl, n-hexadecyl and n-dodecyl; branched and cyclic C₆-C₁₈ alkyl radicals, for example, cyclododecane (C₁₂) and adamantane (C₁₀); C₆-C₁₈ perfluoroalkyl radicals, for example, the group of formula —(CH₂)₂—(CF₂)₉—CF₃; cholesteryl radicals (C₂₇) and cholesterol ester residues, for example, the cholesteryl oxyhexanoate group; and aromatic polycyclic groups, for example, naphthalene and pyrene.

In one embodiment, the unit of formula (II) comprises at least one alkylene oxide unit (x≧1) such as a polyoxyalkylenated chain. The polyoxyalkylenated chain, for example, comprises units chosen from ethylene oxide units and propylene oxide units. In one embodiment, the polyoxyalkylenated chain comprises ethylene oxide units. The number of oxyalkylene units ranges, for example, from 3 to 100, such as from 3 to 50 and further such as from 7 to 25.

Among these polymers, mention may be made of:

crosslinked or noncrosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units, relative to the total weight of the polymer, such as those described in patent application EP-A-750 899; and

terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of non-crosslinked and crosslinked copolymers of partially or totally neutralized AMPS and of dodecyl methacrylate, and non-crosslinked and crosslinked copolymers of partially or totally neutralized AMPS and of n-dodecylmethacrylamide, such as those described in the Morishima articles mentioned above.

Mention may also be made, for example, of copolymers comprising 2-acrylamido-2-methylpropanesulphonic (AMPS) acid units of formula (I) and units of formula (III) below:

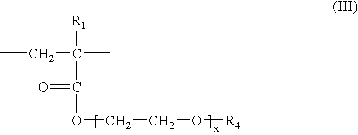

wherein x is an integer ranging from 3 to 100, such as from 5 to 80 and further such as from 7 to 25; R₁ has the same meaning as that given above in formula (II) and R₄ is chosen from linear and branched C₆-C₂₂ alkyl radicals such as linear and branched C₁₀-C₂₂ alkyl radicals.

In one embodiment, the polymers that can be used herein are those for which, in the unit of formula (III), x=25, R₁ is methyl and R₄ is chosen from linear and branched C₁₆-C₁₈ and C₂₂ alkyl radicals.

The at least one amphiphilic polymer disclosed herein may be obtained, for example, according to the standard free-radical polymerization processes in the presence of at least one initiator chosen, for example, from azobisisobutyronitrile (AIBN), azobisdimethylvaleronitrile, ABAH (2,2-azobis[2-amidinopropane]hydrochloride), organic peroxides such as dilauryl peroxide, benzoyl peroxide, tert-butyl hydroperoxide, etc., mineral peroxide compounds such as potassium persulphate and ammonium persulphate, and H₂O₂ optionally in the presence of at least one reducing agent.

The at least one amphiphilic polymer can be obtained, for example, by free-radical polymerization in a tert-butanol medium in which the at least one amphiphilic polymer precipitates.

Using precipitation polymerization in tert-butanol, it is possible to obtain a size distribution of the polymer particles that can be favorable for its uses.

The size distribution of the polymer particles may be determined, for example, by laser diffraction or image analysis.

An example of the distribution for this type of polymer, determined by image analysis, is as follows: 60.2% less than 423 microns, 52.0% less than 212 microns, 26.6% less than 106 microns, 2.6% less than 45 microns and 26.6% greater than 850 microns.

The reaction may be performed at a temperature ranging from 0 to 150° C., such as from 10 to 100° C., either at atmospheric pressure or under reduced pressure. It may also be performed under inert atmosphere, such as under nitrogen.

According to this process, 2-acrylamido-2-methylpropanesulphonic acid (AMPS) or a sodium or ammonium salt thereof was, for example, polymerized with a (meth)acrylic acid ester and at least one of:

a C₁₀-C₁₈ alcohol oxyethylenated with 8 mol of ethylene oxide (Genapol® C-080 from the company Hoechst/Clariant),

a C₁₁ oxo alcohol oxyethylenated with 8 mol of ethylene oxide (Genapol® UD-080 from the company Hoechst/Clariant),

a C₁₁ oxo alcohol oxyethylenated with 7 mol of ethylene oxide (Genapol® UD-070 from the company Hoechst/Clariant),

a C₁₂-C₁₄ alcohol oxyethylenated with 7 mol of ethylene oxide (Genapol® LA-070 from the company Hoechst/Clariant),

a C₁₂-C₁₄ alcohol oxyethylenated with 9 mol of ethylene oxide (Genapol® LA-090 from the company Hoechst/Clariant),

a C₁₂-C₁₄ alcohol oxyethylenated with 11 mol of ethylene oxide (Genapol® LA-110 from the company Hoechst/Clariant),

a C₁₆-C₁₈ alcohol oxyethylenated with 8 mol of ethylene oxide (Genapol® T-080 from the company Hoechst/Clariant),

a C₁₆-C₁₈ alcohol oxyethylenated with 15 mol of ethylene oxide (Genapol® T-150 from the company Hoechst/Clariant),

a C₁₆-C₁₈ alcohol oxyethylenated with 11 mol of ethylene oxide (Genapol® T-110 from the company Hoechst/Clariant),

a C₁₆-C₁₈ alcohol oxyethylenated with 20 mol of ethylene oxide (Genapol® T-200 from the company Hoechst/Clariant),

a C₁₆-C₁₈ alcohol oxyethylenated with 25 mol of ethylene oxide (Genapol® T-250 from the company Hoechst/Clariant),

a C₁₈-C₂₂ alcohol oxyethylenated with 25 mol of ethylene oxide, and

a C₁₆-C₁₈ iso alcohol oxyethylenated with 25 mol of ethylene oxide.

These AMPS copolymers and the process for preparing them have been described, for example, in French Patent Application No. 2 818 543.

The molar % concentrations of the units of formula (II) and of the units of formula (III) in the polymers disclosed herein will vary as a function of the desired cosmetic use and of the desired rheological properties of the formulation, and may range, for example, from 0.1 mol % to 99.9 mol %.

For example, for the most hydrophobic polymers, the molar proportion of units of formula (II) or (III) ranges from 50.1% to 99.9%, such as from 70% to 95% and further such as from 80% to 90%.

In one embodiment, for the sparingly hydrophobic polymers, the molar proportion of units of formula (II) or (III) ranges from 0.1% to 50%, such as from 5% to 25% and further such as from 10% to 20%.

The monomer distribution in the polymers disclosed herein may be, for example, alternating, block (including multiblock) or random.

The viscosities, measured at 25° C. using a Brookfield viscometer, needle No. 7, of the aqueous 1% solutions range, for example, from 20 000 mPa.s to 100 000 mPa.s such as from 60 000 mPa.s to 70 000 mPa.s.

The at least one amphiphilic polymer disclosed herein is present in the oxidizing O/W emulsions in a concentration ranging from 0.01% to 10% by weight, such as from 0.01% to 5% by weight, and further such as from 0.01% to 2% by weight, relative to the total weight of the emulsion.

Oxidizing agent

As disclosed herein, the at least one oxidizing agent is, for example, chosen from hydrogen peroxide and compounds capable of producing hydrogen peroxide by hydrolysis.

For example, the at least one oxidizing agent is chosen from aqueous hydrogen peroxide solution, urea peroxide, alkali metal bromates and ferricyanides and persalts such as perborates and persulphates. At least one redox enzyme chosen, for example, from laccases, peroxidases and 2-electron oxidoreductases such as uricase may also be used as the at least one oxidizing agent, where appropriate in the presence of the respective donor or co-factor thereof.

In one embodiment, the at least one oxidizing agent is hydrogen peroxide, such as an aqueous hydrogen peroxide solution.

The hydrogen peroxide concentration may range from 0.15% to 12% by weight such as from 0.6% to 9% by weight relative to the total weight of the oxidizing emulsion. The concentration of compounds capable of forming hydrogen peroxide by hydrolysis may range from 0.1% to 25% by weight relative to the total weight of the oxidizing emulsion.

In one embodiment, when the at least one oxidizing agent is an aqueous hydrogen peroxide solution, the oxidizing emulsion disclosed herein comprises at least one hydrogen peroxide stabilizer, which may be chosen, for example, from alkali metal and alkaline-earth metal pyrophosphates, alkali metal and alkaline-earth metal stannates, phenacetin and salts of acids and of oxyquinoline, for example, oxyquinoline sulphate. In another embodiment, at least one stannate optionally in combination with at least one pyrophosphate is used.

It is also possible to use salicylic acid and its salts, pyridinedicarboxylic acid and its salts, paracetamol and/or systems comprising a) a buffer, such as ammonium or alkali metal (Na or K) borate and, for example, sodium tetraborate decahydrate, b) an alkaline agent chosen, for example, from NH₄OH, monoethanolamine, ammonium carbonate, ammonium hydrogen carbonate and sodium hydroxide, and c) an agent for sequestering ions of heavy metals, for example, Fe, Mn and Cu, such as those described in patent applications WO 01/72271, WO 01/72272 and WO 01/52801.

In the oxidizing emulsions disclosed herein, the concentration of the at least one hydrogen peroxide stabilizer may range from 0.0001% to 5% by weight such as from 0.01% to 2% by weight relative to the total weight of the oxidizing emulsions.

In the oxidizing emulsions disclosed herein comprising an aqueous hydrogen peroxide solution, the concentration ratio of the hydrogen peroxide to the at least one stabilizer may range from 0.05:1 to 1 000:1, such as from 0.1:1 to 500:1 and further such as from 1:1 to 200:1. Similarly, the concentration ratio of the at least one amphiphilic polymer disclosed herein to the at least one stabilizer may range from 0.05:1 to 1 000:1, such as from 0.1:1 to 500:1 and further such as from 1:1 to 200:1.

In one embodiment, the concentration ratio of the at least one amphiphilic polymer disclosed herein to the at least one oxidizing agent ranges from 0.001:1 to 10:1, wherein the amounts of the at least one amphiphilic polymer and the at least one oxidizing agent are expressed as active materials, for example, hydrogen peroxide for the aqueous hydrogen peroxide solution. In another embodiment, this ratio ranges from 0.01:1 to 5:1 such as from 0.02:1 to 1:1.

C₈-C₃₀ Fatty Alcohols

As disclosed herein, the term “fatty alcohol” means any pure, saturated or unsaturated, linear or branched fatty alcohol.

Among the C₈-C₃₀ fatty alcohols, C₁₂-C₂₂ fatty alcohols are, for example, used.

Mention may be made among these of lauryl alcohol, cetyl alcohol, stearyl alcohol, oleyl alcohol, behenyl alcohol, linoleyl alcohol, undecylenyl alcohol, palmitoleyl alcohol, linolenyl alcohol, arachidonyl alcohol and erucyl alcohol, and mixtures thereof.

In one embodiment, cetyl alcohol is used.

In the oxidizing emulsions disclosed herein, the concentration of the at least one fatty alcohol may range from 0.1% to 30% by weight such as from 0.5% to 15% by weight relative to the total weight of the emulsion.

Nonionic and/or Anionic Surfactants

The nonionic surfactants are compounds that are well known per se (see, for example, “Handbook of Surfactants” by M. R. Porter, published by Blackie & Son (Glasgow and London), 1991, pp. 116-178) and, in the context of the present invention, their nature is not a critical factor. Thus, they may be chosen, for example, from polyethoxylated and polypropoxylated alcohols, α-diols and alkylphenols, with a fatty chain comprising, for example, from 8 to 18 carbon atoms, wherein the number of ethylene oxide or propylene oxide groups possibly ranges, for example, from 2 to 50. Mention may also be made of copolymers of ethylene oxide and of propylene oxide, condensates of ethylene oxide and of propylene oxide with fatty alcohols; polyethoxylated fatty amides, such as those comprising from 2 to 30 mol of ethylene oxide, polyglycerolated fatty amides comprising on average from 1 to 5 such as from 1.5 to 4 glycerol groups; glycerolated fatty alcohols; oxyethylenated fatty acid esters of sorbitan comprising from 2 to 30 mol of ethylene oxide; fatty acid esters of sucrose; fatty acid esters of polyethylene glycol, alkylpolyglycosides, N-alkylglucamine derivatives, and amine oxides such as (C₁₀-C₁₄)alkylamine oxides and N-acylaminopropylmorpholine oxides.

As disclosed herein, among the nonionic surfactants that can, for example, be used are glycerolated fatty alcohols. The glycerolated fatty alcohols are, for example, of the following formula (IV):

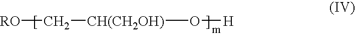

wherein:

R is chosen from saturated and unsaturated, linear and branched radicals comprising from 8 to 40 carbon atoms such as from 10 to 30 carbon atoms;

m is a number ranging from 1 to 30 such as from 1 to 10.

Compounds of this type that may be mentioned include lauryl alcohol containing 4 mol of glycerol (INCI name: Polyglyceryl-4 lauryl ether), lauryl alcohol containing 1.5 mol of glycerol, oleyl alcohol containing 4 mol of glycerol (INCI name: Polyglyceryl-4 oleyl ether), oleyl alcohol containing 2 mol of glycerol (INCI name: Polyglyceryl-2 oleyl ether), cetearyl alcohol containing 2 mol of glycerol, cetearyl alcohol containing 6 mol of glycerol, oleocetyl alcohol containing 6 mol of glycerol and octadecanol containing 6 mol of glycerol.

The fatty alcohol may represent a mixture of fatty alcohols in the same respect that the value of m represents a random value, which means that, in a commercial product, several species of polyglycerolated fatty alcohols may coexist in the form of a mixture.

The anionic surfactants that may be used, alone or as mixtures, include, for example, salts such as alkali metal salts, for example, sodium salts, ammonium salts, amine salts, amino alcohol salts and magnesium salts of the following compounds: alkyl sulphates, alkyl ether sulphates, alkylamido ether sulphates, alkylarylpolyether sulphates and monoglyceride sulphates; alkyl sulphonates, alkyl phosphates, alkylamide sulphonates, alkylaryl sulphonates, α-olefin sulphonates and paraffin sulphonates; (C₆-C₂₄)alkyl sulphosuccinates, (C₆-C₂₄)alkyl ether sulphosuccinates and (C₆-C₂₄)alkylamide sulphosuccinates; (C₆-C₂₄)alkyl sulphoacetates; (C₆-C₂₄)acyl sarcosinates and (C₆-C₂₄)acyl glutamates. It is also possible to use (C₆-C₂₄)alkylpolyglycoside carboxylic esters such as alkylglucoside citrates, alkylpolyglycoside tartrates and alkylpolyglycoside sulphosuccinates, alkyl sulphosuccinamates; acyl isethionates and N-acyl taurates, wherein the alkyl or acyl radical of all these various compounds comprises, for example, from 12 to 20 carbon atoms, and the aryl radical is chosen, for example, from phenyl and benzyl groups. Among the anionic surfactants that may be used, mention may also be made of fatty acid salts such as oleic, ricinoleic, palmitic and stearic acid salts, and coconut oil acids and hydrogenated coconut oil acids; acyl lactylates wherein the acyl radical comprises from 8 to 20 carbon atoms. It is also possible to use alkyl D-galactosideuronic acids and salts thereof, polyoxyalkylenated (C₆-C₂₄)alkyl ether carboxylic acids, polyoxyalkylenated (C₆-C₂₄)alkylaryl ether carboxylic acids and polyoxyalkylenated (C₆-C₂₄)alkylamido ether carboxylic acids and salts thereof, such as those comprising from 2 to 50 alkylene oxide groups and, for example, ethylene oxide groups, and mixtures thereof.

Among the anionic surfactants that can be used herein, mention may be made of, for example, alkyl sulphates, alkyl ether sulphates and α-olefin sulphonates.

Mixtures of nonionic and anionic surfactants are, for example, used.

The at least one surfactant chosen from nonionic and anionic surfactants is present in an amount ranging from 0.1% to 30% by weight such as from 0.5% to 15% by weight relative to the total weight of the emulsion.

The oxidizing O/W cream emulsions disclosed herein have a pH ranging, for example, from 1 to 6, such as from 2 to 4.

The pH of these emulsions disclosed herein may be obtained and/or adjusted conventionally by adding either at least one basifying agent chosen, for example, from aqueous ammonia, monoethanolamine, diethanolamine, triethanolamine, isopropanolamine, 1,3-propanediamine, ammonium and alkali metal carbonates and bicarbonates, organic carbonates such as guanidine carbonate, and alkali metal hydroxides, or at least one acidifying agent, chosen, for example, from hydrochloric acid, phosphoric acid, acetic acid, lactic acid, citric acid, tartaric acid and boric acid.

The O/W oxidizing cream emulsion may also comprise an effective amount of at least one additive chosen from the additives known for their use in oxidizing compositions for the oxidation dyeing of the hair or for bleaching and permanently reshaping the hair, such as preserving agents, sequestering agents such as EDTA, DTPA and etidronic acid, antifoams, for example, simethicone, cationic and amphoteric substantive polymers, water-soluble thickening polymers, fragrances and dyes to color the emulsion.

Needless to say, a person skilled in the art will take care to select the optional additional compound(s) mentioned above, such that the advantageous properties intrinsically associated with the emulsion disclosed herein are not, or are not substantially, adversely affected by the envisaged addition(s).

Further disclosed herein is a process for oxidation dyeing of human keratin fibers such as the hair, comprising applying to the human keratin fibers an oxidizing emulsion as defined above and a dye composition comprising, in a support suitable for dyeing the keratin fibres, at least one oxidation dye.

According to this process, color can be revealed at acidic, neutral or alkaline pH with the aid of an oxidizing emulsion disclosed herein that is applied simultaneously or sequentially, with or without intermediate rinsing. In one embodiment, the dye composition is mixed, at the time of use, with an oxidizing emulsion disclosed herein. The mixture obtained is then applied to the keratin fibers and is left in for a leave-in time ranging from 3 to 50 minutes approximately such as from 5 to 30 minutes approximately, after which the keratin fibers are rinsed, washed with shampoo, rinsed again and dried.

The oxidizing composition disclosed herein may also be used in a process for bleaching human keratin fibers such as the hair.

The bleaching process disclosed herein comprises applying to the keratin fibers an oxidizing emulsion disclosed herein, wherein the oxidizing emulsion comprises, for example, an aqueous hydrogen peroxide solution in alkaline medium after extemporaneous mixing.

The bleaching process disclosed herein may further comprise rinsing the treated keratin fibers.

Even further disclosed herein is a process for permanently reshaping human keratin fibers such as the hair, using the oxidizing emulsion defined above as oxidizing composition.

According to this process, a reducing composition is applied to the keratin fibers to be treated, the keratin fibers are placed under mechanical tension before, during or after the application, the keratin fibers are optionally rinsed, the oxidizing emulsion disclosed herein is applied to the optionally rinsed keratin fibers, and the keratin fibers are then optionally rinsed again.

The first stage of this process comprises applying a reducing composition to the keratin fibers such as the hair. This application can be performed lock by lock or to the whole head of hair.

The reducing composition comprises at least one reducing agent, which may be chosen, for example, from thioglycolic acid, cysteine, cysteamine, glyceryl thioglycolate, thiolactic acid, and salts of thiolactic acid and of thioglycolic acid.

The usual step of placing the hair under tension in a shape corresponding to the final shape desired for the hair, for example, curls, may be performed by any means, such as a mechanical means that is suitable and known per se for keeping the hair under tension, for example, rollers, curlers and the like.

The hair may also be shaped without the aid of external means, simply with the fingers.

After the hair is shaped, and before performing optional rinsing, it is conventionally convenient to leave the head of hair, to which the reducing composition has been applied, at rest for a few minutes, generally ranging from 5 minutes to one hour and such as from 10 to 30 minutes, so to be sure to give the reducing agent the time to act correctly on the hair. This waiting phase may take place at a temperature ranging, for example, from 35° C. to 45° C., and also, for example, with a hood protecting the hair.

If rinsed (rinsing is optional), the hair impregnated with the reducing composition is rinsed thoroughly with an aqueous composition.

Next, the oxidizing emulsion disclosed herein is applied to the hair thus rinsed, with the aim of fixing the new shape given to the hair.

As in the case of the application of the reducing composition, the head of hair to which the oxidizing emulsion has been applied can then be conventionally left in a resting or waiting phase that lasts a few minutes, generally ranging from 3 to 30 minutes such as from 5 to 15 minutes.

If the hair was maintained under tension by external means, these means, such as rollers, curlers and the like, may be removed from the hair before or after the fixing operation.

Finally, the hair impregnated with the oxidizing composition is optionally rinsed thoroughly, generally with water.

The examples that follow illustrate the invention without being limiting in nature.

### EXAMPLES

The two oxidizing compositions A and B below were prepared (amounts expressed in grams of Active Material (AM*))

| | Composition A | Composition B |
|---|---|---|
| | not according to the | according to |
| | invention | the invention |
| Cetyl alcohol | 3 | 3 |
| Sodium lauryl sulphate | 0.5 | 0.5 |
| Oleyl alcohol glycerolated | 0.45 | 0.45 |
| with 2 mol of glycerol | | |
| Oleyl alcohol glycerolated | 0.35 | 0.35 |
| with 4 mol of glycerol | | |
| Antifoam: Simethicone | 0.045 | 0.045 |
| Sequestering agent: DTPA | 0.06 | 0.06 |
| Tetrasodium pyrophosphate | 0.02 | 0.02 |
| 10 H₂O | | |
| Sodium stannate, 6 H₂O | 0.04 | 0.04 |
| Amphiphilic polymer according | | 0.05 |
| to the invention** | | |
| Aqueous 50% hydrogen | 12 AM* | 12 AM* |
| peroxide solution | | |
| Aqueous 85% phosphoric acid | 3 | 3 |
| solution qs pH | | |
| Demineralized water qs | 100 | 100 |

**Copolymer of AMPS (80)/C₁₆-C₁₈ alkyl methacrylate oxyethylenated with 25 mol of ethylene oxide (20), crosslinked with TMPTA, prepared and described in French Patent Application No. 2 818 543.

The change in the texture/viscosity of the two compositions A and B were monitored for seven weeks.

To quantify the change in the texture over time, the parameter Δv has been defined.

Change in the texture over time: Δv=viscosity measured at time t-viscosity measured 24 hours after manufacture.

The viscosities were measured using a rheomat, at a temperature of 25° C. and with a No. 2 spindle. They are expressed as mean±standard deviation.

| | Mean over 3 manufactures (1 measurement | | | | |
|---|---|---|---|---|---|
| Time after | per manufacture) ± standard deviation | | | | |
| manufacture | 1 week | 2 weeks | 3 weeks | 4 weeks | 7 weeks |
| Δv (A) | 5.0 ± 1.2 | 10.0 ± 2.3 | 15.0 ± 2.6 | 18.0 ± 2.2 | −5.0 ± 2.1 |
| Δv (B) | 2.6 ± 0.9 | 2.6 ± 0.8 | 4.6 ± 1.2 | 6.0 ± 1.3 | 7.0 ± 1.2 |

These results indicate that the viscosity of composition B, which is in accordance with the invention, changes significantly less over time than composition A, which is not in accordance with the invention. In addition, it was found that composition B showed better stability and would therefore maintain its use qualities better over time for use in dyeing, bleaching or permanently reshaping the hair.

After 7 weeks, composition B according to the invention also remained stable with respect to the aqueous hydrogen peroxide solution.

What is claimed is:

1. An oxidizing cream oil-in-water emulsion for treating a keratin material, comprising:
at least one oxidizing agent,
at least one fatty alcohol chosen from (C₈-C₃₀) fatty alcohols,
at least one surfactant chosen from nonionic and anionic surfactants and
at least one amphiphilic polymer comprising
at least one 2-acrylamido-2-methylpropanesulphonic acid unit in free form or partially or totally neutralized form of formula (I) below and
at least one hydrophobic unit comprising from 6 to 50 carbon atoms, 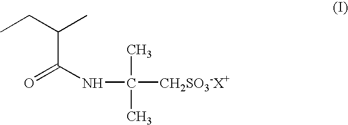
wherein X⁺ is chosen from a proton, alkali metal cations, alkaline-earth metal cations and an ammonium ion.

2. The emulsion according to claim 1, wherein the at least one hydrophobic unit of the at least one amphiphilic polymer comprises from 12 to 22 carbon atoms.

3. The emulsion according to claim 1, wherein the at least one amphiphilic polymer has a number-average molecular weight ranging from 1 000 to 20 000 000 g/mol.

4. The emulsion according to claim 3, wherein the at least one amphiphilic polymer has a number-average molecular weight ranging from 20 000 to 5 000 000 g/mol.

5. The emulsion according to claim 4, wherein the at least one amphiphilic polymer has a number-average molecular weight ranging from 100 000 to 1 500 000 g/mol.

6. The emulsion according to claim 1, wherein an aqueous 1% by weight solution of the at least one amphiphilic polymer has, at a temperature of 25° C., a viscosity measured using a Brookfield viscometer, No. 7 needle, ranging from 20 000 mPa.s to 100 000 mPa.s.

7. The emulsion according to claim 1, wherein the at least one amphiphilic polymer is chosen from the amphiphilic polymers prepared by free-radical polymerization by precipitation in tert-butanol.

8. The emulsion according to claim 1, wherein the at least one amphiphilic polymer is chosen from non-crosslinked amphiphilic polymers and crosslinked amphiphilic polymers, wherein said crosslinked amphiphilic polymers are crosslinked with at least one crosslinking agent.

9. The emulsion according to claim 8, wherein the at least one amphiphilic polymer is chosen from the crosslinked amphiphilic polymers.

10. The emulsion according to claim 8, wherein the at least one crosslinking agent is chosen from polyolefinically unsaturated compounds.

11. The emulsion according to claim 10, wherein the at least one crosslinking agent is chosen from methylenebisacrylamide, allyl methacrylate and trimethylolpropane triacrylate (TMPTA).

12. The emulsion according to claim 1, wherein the at least one hydrophobic unit is chosen from acrylates and acrylamides of formula (II) below: 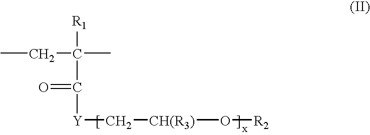
wherein
R₁ and R₃, which may be identical or different, are each chosen from a hydrogen atom and linear and branched C₁-C₆ alkyl radicals;
Y is chosen from O and NH;
R₂ is a hydrophobic hydrocarbon-based radical chosen from those comprising from 6 to 50 carbon atoms;
x is a number of moles of alkylene oxide (oxyalkylene) and ranges from 0 to 100.

13. The emulsion according to claim 12, wherein, in defining R₁ and R₃ in the formula (II), the linear and branched C₁-C₆ alkyl radicals are chosen from a methyl radical.

14. The emulsion according to claim 12, wherein the hydrophobic hydrocarbon-based radical R₂ is chosen from those comprising from 12 to 22 carbon atoms.

15. The emulsion according to claim 12, wherein the hydrophobic hydrocarbon-based radical R₂ is chosen from linear, branched and cyclic C₆-C₁₈ alkyl radicals; C₆-C₁₈ perfluoroalkyl radicals; cholesteryl radicals and cholesterol ester residues; and aromatic polycyclic groups.

16. The emulsion according to claim 12, wherein the at least one hydrophobic unit chosen from acrylates and acrylamides of formula (II) comprises at least one oxyalkylene unit (x≧1).

17. The emulsion according to claim 12, wherein the at least one hydrophobic unit chosen from acrylates and acrylamides of formula (II) comprises at least one polyoxyalkylenated chain.

18. The emulsion according to claim 17, wherein the at least one polyoxyalkylenated chain comprises units chosen from ethylene oxide units and propylene oxide units.

19. The emulsion according to claim 18, wherein the at least one polyoxyalkylenated chain consists of ethylene oxide units.

20. The emulsion according to claim 12, wherein the number of oxyalkylene units ranges from 3 to 100.

21. The emulsion according to claim 20, wherein the number of oxyalkylene units ranges from 3 to 50.

22. The emulsion according to claim 21, wherein the number of oxyalkylene units ranges from 7 to 25.

23. The emulsion according to claim 12, wherein the at least one amphiphilic polymer is chosen from:
crosslinked and noncrosslinked, neutralized and non-neutralized copolymers comprising from 15% to 60% by weight of the at least one 2-acrylamido-2-methylpropane-sulphonic acid unit and from 40% to 85% by weight of the at least one hydrophobic unit chosen from (C₈-C₁₆)alkyl(meth)acrylamide units and (C₈-C₁₆)alkyl (meth)acrylate units, relative to the total weight of the at least one amphiphilic polymer; and
terpolymers comprising from 10 mol % to 90 mol % of acrylamide units, from 0.1 mol % to 10 mol % of the at least one 2-acrylamido-2-methylpropanesulphonic acid unit and from 5 mol % to 80 mol % of n-(C₆-C₁₈)alkylacrylamide units, relative to the total weight of the at least one amphiphilic polymer.

24. The emulsion according to claim 12, wherein the at least one amphiphilic polymer comprising the at least one 2-acrylamido-2-methylpropanesulphonic acid unit in free form or partially or totally neutralized form of formula (I) and the at least one hydrophobic unit comprising from 6 to 50 carbon atoms is chosen from:
non-crosslinked and crosslinked copolymers of the at least one 2-acrylamido-2-methylpropanesulphonic acid unit and of n-dodecyl methacrylate; and
non-crosslinked and crosslinked copolymers of the at least one 2-acrylamido-2-methylpropanesulphonic acid unit and of n-dodecylmethacrylamide.

25. The emulsion according to claim 12, wherein the at least one amphiphilic polymer is chosen from copolymers comprising the at least one 2-acrylamido-2-methylpropanesulphonic acid unit in free form or partially or totally neutralized form of formula (I) and at least one unit chosen from the units of formula (III) below: 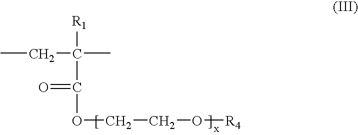
wherein
x is an integer ranging from 3 to 100;
R₁ has the same meaning as that given in the formula (II); and
R₄ is chosen from linear and branched C₆-C₂₂ alkyl radicals.

26. The emulsion according to claim 25, wherein, in the formula (III), r is an integer ranging from 5 to 80.

27. The emulsion according to claim 26, wherein, in formula (III), r is an integer ranging from 7 to 25.

28. The emulsion according to claim 25, wherein, in formula (III), R₄ is chosen from linear and branched C₁₀-C₂₂ alkyl radicals.

29. The emulsion according to claim 25, wherein x=25, R₁ is methyl and R₄ is chosen from linear and branched C₁₆-C₁₈ and C₂₂ alkyl radicals.

30. The emulsion according to claim 12, wherein the molar percentage proportion of the units of the formula (II) in the at least one amphiphilic polymer ranges from 50.1% to 99.9%.

31. The emulsion according to claim 25, wherein the molar percentage proportion of the units of the formula (III) in the at least one amphiphilic polymer ranges from 50.1% to 99.9%.

32. The emulsion according to claim 12, wherein the molar percentage proportion of the units of the formula (II) in the at least one amphiphilic polymer ranges from 0.1% to 50%.

33. The emulsion according to claim 25, wherein the molar percentage proportion of the units of the formula (III) in the at least one amphiphilic polymer ranges from 0.1% to 50%.

34. The emulsion according to claim 1, wherein the at least one amphiphilic polymer is present in a concentration ranging from 0.01% to 10% by weight relative to the total weight of the emulsion.

35. The emulsion according to claim 34, wherein the at least one amphiphilic polymer is present in a concentration ranging from 0.01% to 5% by weight relative to the total weight of the emulsion.

36. The emulsion according to claim 35, wherein the at least one amphiphilic polymer is present in a concentration ranging from 0.01% to 2% by weight relative to the total weight of the emulsion.

37. The emulsion according to claim 1, wherein the at least one oxidizing agent is chosen from hydrogen peroxide, urea peroxide, alkali metal bromates and ferricyanides, persalts, and redox enzymes where appropriate in the presence of the respective donor or co-factor thereof.

38. The emulsion according to claim 37, wherein the redox enzymes are chosen from laccases, peroxidases and 2-electron oxidoreductases.

39. The emulsion according to claim 37, wherein the at least one oxidizing agent is hydrogen peroxide.

40. The emulsion according to claim 39, wherein the hydrogen peroxide is present in a concentration ranging from 0.15% to 12% by weight relative to the total weight of the emulsion.

41. The emulsion according to claim 40, wherein the hydrogen peroxide is present in a concentration ranging from 0.6% to 9% by weight relative to the total weight of the emulsion.

42. The emulsion according to claim 39, wherein the at least one oxidizing agent is an aqueous hydrogen peroxide solution.

43. The emulsion according to claim 1, wherein the oxidizing agent is present in a concentration ranging from 0.1% to 25% by weight relative to the total weight of the emulsion.

44. The emulsion according to claim 1, wherein the emulsion has a pH ranging from 1 to 6.

45. The emulsion according to claim 44, wherein the emulsion has a pH ranging from 2 to 4.

46. The emulsion according to claim 1, wherein the at least one fatty alcohol is chosen from C₁₂-C₂₂ fatty alcohols.

47. The emulsion according to claim 46, wherein the at least one fatty alcohol is cetyl alcohol.

48. The emulsion according to claim 1, wherein the at least one fatty alcohol is present in a concentration ranging from 0.1% to 30% by weight relative to the total weight of the emulsion.

49. The emulsion according to claim 48, wherein the at least one fatty alcohol is present in a concentration ranging from 0.5% to 15% by weight relative to the total weight of the emulsion

50. The emulsion according to claim 1, wherein the anionic surfactants are chosen from alkyl sulphates, alkyl ether sulphates and α-olefin sulphonates.

51. The emulsion according to claim 1, wherein the nonionic surfactants are chosen from glycerolated fatty alcohols.

52. The emulsion according to claim 1, wherein the at least one surfactant is chosen from mixtures of nonionic and anionic surfactants.

53. The emulsion according to claim 1, wherein the at least one surfactant is present in a concentration ranging from 0.1% to 30% by weight relative to the total weight of the emulsion.

54. The emulsion according to claim 53, wherein the at least one surfactant is present in a concentration ranging from 0.5% to 15% by weight relative to the total weight of the emulsion.

55. The emulsion according to claim 1, wherein the keratin material is human keratin fibers.

56. The emulsion according to claim 55, wherein the human keratin fibers are hair.

57. The emulsion according to claim 42, further comprising at least one hydrogen peroxide stabilizer.

58. The emulsion according to claim 57, wherein the at least one hydrogen peroxide stabilizer is present in a concentration ranging from 0.0001% to 5% by weight relative to the total weight of the emulsion.

59. The emulsion according to claim 1, further comprising at least one additive.

60. A process for oxidation dyeing of human keratin fibers, comprising applying to the keratin fibers
a dye composition comprising, in a support suitable for dyeing the keratin fibers, at least one oxidation dye; and
an oxidizing emulsion comprising at least one oxidizing agent, at least one fatty alcohol chosen from (C₈-C₃₀) fatty alcohols, at least one surfactant chosen from nonionic and anionic surfactants and at least one amphiphilic polymer comprising at least one 2-acrylamido-2-methylpropanesulphonic acid unit in free form or partially or totally neutralized form of formula (I) below and at least one hydrophobic unit comprising from 6 to 50 carbon atoms, 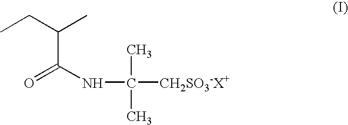
wherein X⁺ is chosen from a proton, alkali metal cations, alkaline-earth metal cations and an ammonium ion.

61. The process according to claim 60, wherein the human keratin fibers are hair.

62. A process for oxidation dyeing of human keratin fibers, comprising
mixing a dye composition, at the time of use, with an oxidizing emulsion, wherein
said dye composition comprises, in a support suitable for dyeing the keratin fibers, at least one oxidation dye; and
said oxidizing emulsion comprises at least one oxidizing agent, at least one fatty alcohol chosen from (C₈-C₃₀) fatty alcohols, at least one surfactant chosen from nonionic and anionic surfactants and at least one amphiphilic polymer comprising at least one 2-acrylamido-2-methylpropanesulphonic acid unit in free form or partially or totally neutralized form of formula (I) below and at least one hydrophobic unit comprising from 6 to 50 carbon atoms, 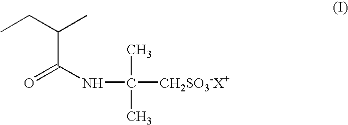
wherein X⁺ is chosen from a proton, alkali metal cations, alkaline-earth metal cations and an ammonium ion
applying the mixture obtained to the keratin fibres,
leaving the mixture on the keratin fibers for a leave-in time ranging from 3 to 50 minutes; and
optionally rinsing the keratin fibers,
optionally washing the keratin fibers with shampoo,
optionally rinsing said fibers again, and
optionally drying the keratin fibers.

63. The process according to claim 62, wherein the leave-in time ranges from 5 to 30 minutes.

64. The process according to claim 60, wherein the dye composition and the oxidizing emulsion are applied sequentially, with or without intermediate rinsing.

65. A process for treating human keratin fibers in order to permanently reshape the keratin fibers, comprising:
(i) applying to the keratin fibers a reducing composition, wherein the keratin fibers are placed under mechanical tension before, during or after said application,
(ii) optionally rinsing the keratin fibers,
(iii) applying to the optionally rinsed keratin fibers an oxidizing emulsion comprising at least one oxidizing agent, at least one fatty alcohol chosen from (C₈-C₃₀) fatty alcohols, at least one surfactant chosen from nonionic and anionic surfactants and at least one amphiphilic polymer comprising at least one 2-acrylamido-2-methylpropanesulphonic acid unit in free form or partially or totally neutralized form of formula (I) below and at least one hydrophobic unit comprising from 6 to 50 carbon atoms, 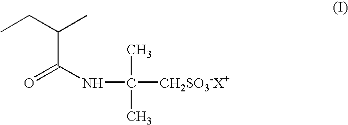
wherein X⁺ is chosen from a proton, alkali metal cations, alkaline-earth metal cations and an ammonium ion, and
(iv) optionally rinsing again the keratin fibers.

66. The process according to claim 65, wherein the human keratin fibers are hair.

67. The process according to claim 66, wherein the keratin fibers are permanently reshaped in the form of permanent-waved hair.

68. A process for bleaching human keratin fibers, comprising:
(i) applying to the keratin fibers an oxidizing emulsion comprising at least one oxidizing agent, at least one fatty alcohol chosen from (C₈-C₃₀) fatty alcohols, at least one surfactant chosen from nonionic and anionic surfactants and at least one amphiphilic polymer comprising at least one 2-acrylamido-2-methylpropanesulphonic acid unit in free form or partially or totally neutralized form of formula (I) below and at least one hydrophobic unit comprising from 6 to 50 carbon atoms, 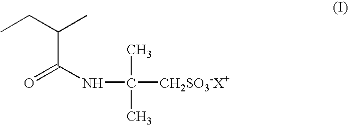
wherein X⁺ is chosen from a proton, alkali metal cations, alkaline-earth metal cations and an ammonium ion, and
(ii) rinsing the keratin fibers thus treated.

69. The process according to claim 68, wherein the human keratin fibers are hair.

70. A method for stabilizing the viscosity of an oxidizing oil-in-water emulsion, comprising including in the emulsion at least one amphiphilic polymer comprising at least one 2-acrylamido-2-methylpropanesulphonic acid unit in free form or partially or totally neutralized form of formula (I) below and at least one hydrophobic unit comprising from 6 to 50 carbon atoms, 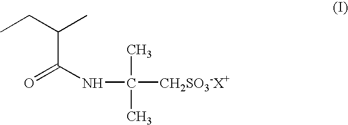
wherein X⁺ is chosen from a proton, alkali metal cations, alkaline-earth metal cations and an ammonium ion,
wherein the emulsion comprises at least one fatty alcohol and at least one surfactant chosen from nonionic and anionic surfactants.

71. The method according to claim 70, wherein the at least one fatty alcohol is chosen from (C₈-C₃₀) fatty alcohols.
